(19) United States
(12) Patent Application Publication
(10) Pub. No.: US 2026/0118335 A1
(43) Pub. Date: Apr. 30, 2026

(54) SYSTEM AND METHOD OF BUMP TESTING IN A MONITORING PLATFORM

(71) Applicant: Industrial Scientific Corporation, Pittsburgh, PA (US)

(72) Inventors: Tae Yeon Won, Mars, PA (US); Laura Jean Faust, Pittsburgh, PA (US); Jonathan Eric Dinsmore, Aliquippa, PA (US); Justin Allen Bee, Follansbee, WV (US); Grayham Matthew Grega, Imperial, PA (US); Ajaykumar Raju Bhupathiraju, McDonald, PA (US); Alan Scott Lofstead, Canonsburg, PA (US); Lisa Ann McCracken, Oakdale, PA (US); Joshua Lazzari, Canonsburg, PA (US); Sachin Shah, Troy, MI (US)

(21) Appl. No.: 19/400,915

(22) Filed: Nov. 25, 2025

Related U.S. Application Data
(63) Continuation of application No. PCT/US2025/040942, filed on Aug. 6, 2025.
(60) Provisional application No. 63/712,661, filed on Oct. 28, 2024.

(51) Int. Cl. G01N 33/00

(52) U.S. Cl. CPC G01N 33/0072; G01N 33/0031; G01N 33/0062; G01N 33/0075

(57) ABSTRACT

A system for gas monitoring includes a cooperative monitoring group for gas monitoring including a plurality of gas monitors comprising a gas sensing element and a wireless communication interface, a gas station, and a data manager. The gas station includes at least one gas monitor physical interface comprising a gas coupling, a wireless communication interface, and a test manager configured to perform a sensing element response test of the gas sensing element. The data manager includes a wireless communication interface and an external communication interface. The cooperative monitoring group is configured to operate as a mesh network, and external communications of the cooperative monitoring group from the plurality of gas monitors and the gas station are routed through the data manager.

### CROSS-REFERENCE TO RELATED APPLICATIONS

This application claims priority to and is a continuation of International Patent Application Serial No. PCT/US2025/040942, filed Aug. 6, 2025 (Attorney Docket No. ISCI-0057-WO).

International Patent Application Serial No. PCT/US2025/040942 claims the benefit of and priority to U.S. Application Ser. No. 63/712,661, filed on Oct. 28, 2024 (Attorney Docket No. ISCI-0056-P01).

Each of the foregoing applications is incorporated herein by reference in its entirety for all purposes.

### BACKGROUND

Gas monitoring at various types of industrial facilities is crucial to maintaining a safe environment, protecting workers, and ensuring proper operation of facilities. Previously known gas monitoring systems suffer from a number of drawbacks. For example, increasing electronic demands on monitoring devices compete with the desire for long battery life, and reduction of time spent on power management of gas monitors. In another example, devices need to be calibrated and maintained, and the workflows to perform these operations are burdensome to the ongoing operation of the facility and for individual operators using a gas monitor. Previously known systems do not provide for convenient and ready monitoring environments that can support arbitrary monitoring, for example in locations such as enclosed spaces, that have high and convenient availability, and that are integrated into the general alarm system for the gas monitoring system. Further, previously known systems are challenged by the flexibility required to support different industrial facilities with different constraints and implementation challenges, where significant challenges are raised in designing systems to mee the needs of some facilities without forcing excessive cost into the solution for features that are not needed.

### SUMMARY

The disclosure herein provides numerous benefits with respect to addressing the challenges of efficiently managing gas monitoring operations in an industrial facility. A person of skill in the art having the benefit of this disclosure will understand that the disclosed embodiments are especially suited for providing firmware execution for instruments in a monitoring platform (e.g., firmware can be chunked for download and implemented at an appropriate time), operating unpowered anchors for range extension (e.g., anchors have long battery life, can be integrated into a network operating as a beacon or fence-line member), improved wireless instrument charging, improved bump testing at gas stations, and improved situational interactions (e.g., alarm thresholds can be changed based on PPE that the person is wearing, or monitors can couple to a smart mask to get its parameters and provide guidance on proper placement). Other benefits may also be present, and embodiments disclosed herein may not have certain or all of the described benefits.

### BRIEF DESCRIPTION OF THE FIGURES

FIG. 1 schematically depicts an example gas monitoring system for an industrial facility.

FIG. 2 schematically depicts an example gas monitoring system for an industrial facility.

FIG. 3 schematically depicts an example gas station in external communication.

FIG. 4 schematically depicts an example gas monitoring system for an industrial facility.

FIG. 5 schematically depicts an example gas monitoring system for an industrial facility.

FIG. 6 schematically depicts an example gas monitoring system for an industrial facility.

FIG. 7 schematically depicts an example gas monitoring system with a monitoring device currently depicted off location.

FIG. 8 depicts a number of example monitoring devices.

FIG. 9 depicts a number of example monitoring devices.

FIG. 10 depicts a gas station component, having two different monitor components docked thereon.

FIG. 11 depicts operations for firmware execution for instruments in a monitoring system.

FIG. 12 depicts aspects of the maintenance time limit.

FIG. 13 depicts an example system including unpowered anchors for range extension.

FIG. 14 depicts aspects of the power source.

FIG. 15 depicts a procedure for applying firmware.

FIG. 16 depicts a procedure for applying firmware.

FIG. 17 depicts an anchor bridging a first portion of the mesh network with a second portion of the mesh network.

FIG. 18 depicts aspects of a target location.

FIG. 19 depicts an operation for routing external communications of a cooperative monitoring group.

FIG. 20 depicts a system for gas monitoring that is capable of supporting wireless instrument charging.

FIG. 21 depicts an example gas station.

FIG. 22 depicts an example docking communication interface.

FIG. 23 depicts a procedure for wirelessly charging a gas monitor battery

FIG. 24 depicts an example system for gas monitoring with improved bump testing.

FIG. 25 depicts an operation of performing a sensing element response test of a gas sensing element of a gas monitor.

FIG. 26 depicts an example system configured to address situational interaction.

FIG. 27 depicts an example monitoring circumstance value.

FIG. 28 depicts an example operator procedure value.

FIG. 29 an operation of adjusting a monitoring operational value in response to an interpreted monitoring circumstance value, and an operation of adjusting monitoring operations for a gas constituent in response to the monitoring operational value.

### DETAILED DESCRIPTION

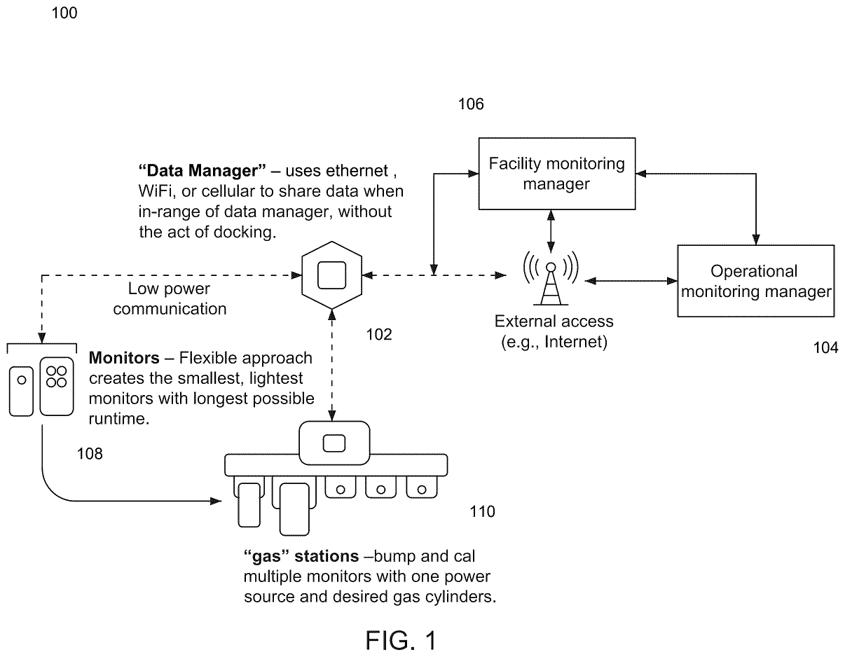

Referencing FIG. 1, an example gas monitoring system 100 for an industrial facility is schematically depicted. The example system utilizes multiple classes of participants on a network supporting the gas monitoring system. The utilization of multiple classes of participants allows for certain participants to be configured to meet the needs of that participant class to support the gas monitoring system, while minimizing the costs and implementation complications introduced by previously known systems, modular systems, or uniquely configured systems.

An example participant class includes at least a data manager class component 102 that manages external communications for the system (e.g., communications to the operational monitoring manager 104 and/or to the facility monitoring manager 106, in the example of FIG. 1). External communications, as utilized herein, include any communications that go to devices apart from the facility, and/or apart from a portion of the facility having the monitored aspects (e.g., where the industrial process that is being monitored is located). External communications may include communications performed using cellular communications, satellite communications, internet communications, wide area network communications, LAN, cloud, or the like. Various embodiments of the present disclosure include other device classes that may have external communications capability, and/or the data manager class component has additional capabilities in certain embodiments.

Another example participant class includes at least a monitoring class component 108 that monitors at least one value in the physical location of the monitor. In certain embodiments, the monitoring class component includes a gas monitor that is responsive to at least one gas species, but other types of monitors, for example including monitors for temperature, radiation, noise/vibration, radiation, or other aspects of interest in the environment are contemplated herein. In certain embodiments, a typical utilization of the monitoring class component is an operator at the facility that carries the monitor on their person. In certain embodiments, a monitoring class component may be positioned at a location for location based monitoring rather than, or in addition to, personal monitoring.

Another example participant class includes a gas station class component 110 (or a docking station class component). The example gas station class component provides support for the monitoring class component(s), and can manage calibration operations and/or rationality checks (e.g., performing a bump test to ensure monitor operation) for the monitoring class components. In certain embodiment, the gas station class component provides for charging operations and/or charge management for monitoring class components. In certain embodiments, a separate charging class component may be provided in the system, for example allowing gas monitors to be charged separately from the gas station class component, and/or as a part of the gas station class component.

Another example participant class includes an anchor class component. The anchor class component provides for extension of the low power network to reach devices, to continue connectivity through a selected area of the facility, and/or to improve availability of communication support in selected locations, challenging areas, for temporary operations, or the like. In certain embodiments, the class scheme provides for limited power requirements from an anchor class component to allow the anchor component to perform operations for an extended period, for example up to three years, operating off of only battery power. Accordingly, anchor placement at a facility does not require consideration of available infrastructure (e.g., power) or installation of additional infrastructure to support monitoring operations. Accordingly, monitoring support for a facility can be rapidly designed and installed with a high confidence of success. Further, monitoring support for a facility can rapidly change with the facility, and grow with the facility, without the need for a redesign. Further, scaling of monitoring operations can be performed by adding to the system, without having to replace base infrastructure or components that are already in place.

The description herein utilizing component classes for a monitoring facility is non-limiting and provided to illustrate certain aspects of the disclosure. A given facility utilizes at least a data manager class component and a number of monitoring class components. The other classes are optional and non-limiting, and may be included according to the goals of the system, including the monitored facility size, number of operators, distribution of operators and/or monitored areas throughout the monitored facility, economic priorities of the operating facility (including, e.g., operational costs, capital expenditures, labor costs, etc.), and/or the monitoring needs of the facility (e.g., personal detection vs. area detection, parameters monitored). Embodiments herein also provide for rapid configuration of the monitoring system to support changes in the monitoring needs of the facility, including operational changes that may be temporary or long term.

Further, a given component may have additional capabilities beyond the baseline for the class, and a given component may have sufficient capabilities to be considered within more than one class. For example, an anchor class component may have a sensor thereon, and could be considered as either an anchor class component or a monitoring class component. In another example, a monitoring class component may have a cellular connection capability, and could be considered as an anchor class component, a monitoring class component, and/or a data manager class component. In certain embodiments, the class of a specific component may depend upon the specific configuration of the component, the operating condition of the facility, the way the component is being utilized, and/or the specific operations being performed. For example, a monitoring component may be utilized as an anchor, and be treated as an anchor class component during that use. The description herein describing components as in particular classes is provided to illustrate aspects of the present disclosure, but it will be understood that a given component may be considered in one class for a first system and in a second class for a second system, and/or the given component may be considered in one class for a first operation, and in a second class for a second operation.

The example of FIG. 1 utilizes an operational monitoring manager, for example that allows an external user to ensure that all devices (e.g., monitoring class components) are in a known location (and/or it is expected that the location may not be presently known), and/or to gather performance and operational data from the gas monitoring system (e.g., collecting monitored data; compliance data such as calibration, bump, or utilization operations; alarms, pre-alarms, and/or other events; performance data relating to the low power network, etc.). In certain embodiments, the operational monitoring manager is embodied on a cloud server at least intermittently in communication with the gas monitoring system (e.g., through the data manager component), and may be operated by a provider of the gas monitoring system. The example of FIG. 1 utilizes a facility monitoring manager, for example that allows someone related to the facility (e.g., a safety office, compliance officer, facility manager, etc.) to receive just the desired information, potentially through an interface set up by and/or configured by someone utilizing the operational monitoring manager. In certain embodiments, the functions of the facility monitoring manager and/or the operational monitoring manager may be combined into a same unit, and/or may be further divided. The facility and/or operational monitoring manager(s) may be positioned separate from the facility, in a separate location on the facility, and/or otherwise remotely positioned in any selected location that is at least intermittently communicatively coupled to the gas monitoring system.

Example features of a “data manager”, “monitor”, and “gas station” are depicted on FIG. 1. Any such descriptions are a non-limiting example. In the example of FIG. 1, the data manager provides external data communication for the gas station, for example ensuring that firmware on the gas station is up to date, and executing operations for updates as indicated. Further, the data manager performs firmware updates for monitoring devices, and/or ensures that the gas station has up to date firmware available for relevant devices. In certain embodiments, gas monitors provide communications with the data manager over the low power network when the gas monitor is within range of the data manager. For example, confirmation of operation, any alerts, alarms, or anomalous readings that the gas monitor has encountered since the last data synch with the data manager, or the like. In certain embodiments, the data manager directly checks the firmware and/or calibration status of the gas monitors. In certain embodiments, checks for proper firmware and/or calibration status of the gas monitors is performed, additionally or alternatively, by the gas station component.

Further in the example of FIG. 1, the gas station provides support for “bump” and “cal” operations. As used herein, a bump operation includes any type of rationality check to ensure that the gas monitor is responsive, for example providing the gas monitor with a plug of a specified gas, and/or a step change in a specified gas, and observing the monitor to ensure that an expected response occurs. As used herein, a calibration operation includes any type of adjustment to the fundamental detection relationship for the gas monitor, for example including updates to any A/D processing (e.g., filters, cut-offs, offsets, scaling, etc.), changes to any models utilized, and/or changes to any coefficients utilized by such models. In certain embodiments, calibration changes may be made in response to any changes in the base models, sensor drift, sensor wear, other changes in the gas monitor over time and/or utilization, and/or changes in the facility—for example facility changes that may indicate that a different model or model settings should be utilized (e.g., facility changes may lead to distinct environments that may affect gas monitoring operations, for example changes in airflow patterns, ambient temperature, background gas constituency, etc.). In certain embodiments, the gas station provides full physical support for bump/cal operations, including for example flowing selected amounts of selected gases through the sensor to support operations. In certain embodiments, the gas station can support multiple monitors mounted thereon, for example at one of a number of mounting locations. In certain embodiments, for example depending upon the number and mix of sensors at the location, the mounting locations may be of varying support configurations—for example a gas station may have five docking locations for supporting a single-gas detection monitor, and two additional docking locations for supporting a four-gas detection monitor. The example gas station performs the bump and cal operations (potentially with support from the data manager as set forth preceding), which is performed automatically in response to the docking operation and frees up the monitoring device for the user (e.g., to store, begin a shift, place on a charger, etc.). In certain embodiments, the gas station may optionally charge the monitoring class device while docked, may have separate charging docks, may have a charging area (e.g., a flat area configured for wireless charging), and/or charging operations for monitoring devices may be provided separately (e.g., a charger positioned in a storage area, etc.).

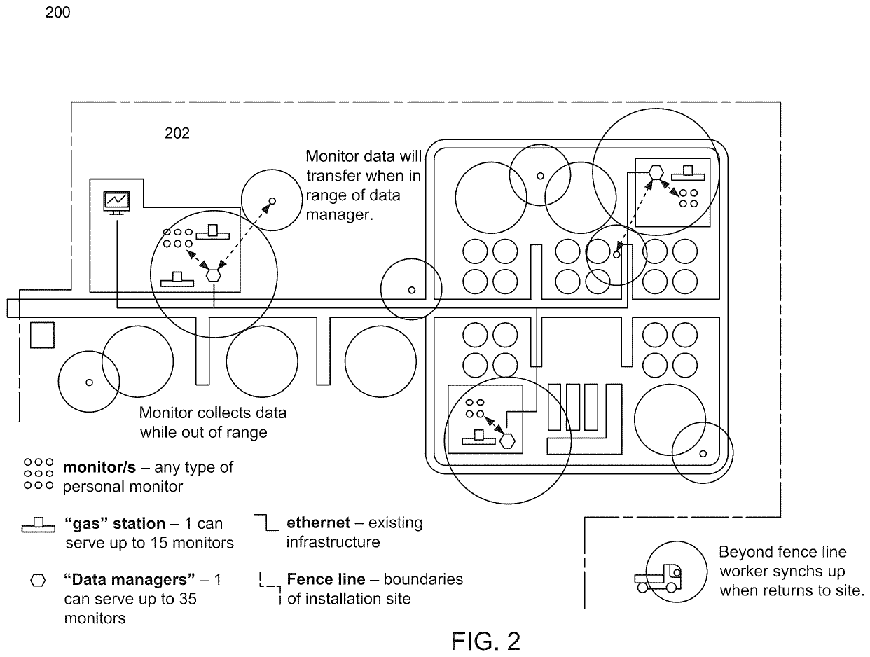

Referencing FIG. 2, another example gas monitoring system 200 includes a facility having a number of gas monitors that have a short range low power network capability. The example gas monitoring system utilizes 100 personal monitors for personnel operating the facility, supported by three data managers and four gas stations, and it should be understood that there is no limitation as to how many monitors a gas station can support. In the example of FIG. 2, the selected data managers are provided according to, for example, the typical locations of personnel to ensure they are in proximity of a data manager during a shift. In the example of FIG. 2, the selected gas stations are provided for sufficient operator support so operational impact of bump/cal operations is minimized. In the example of FIG. 2, two gas stations are co-located in the upper left portion, based on for example the level of operator activity at that location. In the example of FIG. 2, each one of the gas monitors has low power network capability, and utilizes the data manager to tie into the network and communicate with external devices. In the example of FIG. 2, the data managers utilize an ethernet network for on-site communication and coordination, and a lead one, or all, of the data managers can further communicate with external devices utilizing an internet connection, cellular, satellite, or the like. In the example of FIG. 2, monitoring devices in proximity to a data manager can transfer monitor data, receive firmware, receive messages and alerts, or the like from the data manager. In the example of FIG. 2, monitoring devices outside of the proximity of a data manager can link in to the low power network through other monitoring devices in proximity to the device, and/or through anchors or repeaters. In certain embodiments, activity on the low power network for a monitoring device may be limited depending upon how it is connected to the network—for example peer connections (e.g., between monitoring devices) may be limited to alerts, status notifications, passing priority messages, or the like, with other operations such as communicating monitored data, updating firmware, or the like limited to connections with a data manager or as otherwise described herein. A fence line 202 is shown indicating the boundaries of the installation site.

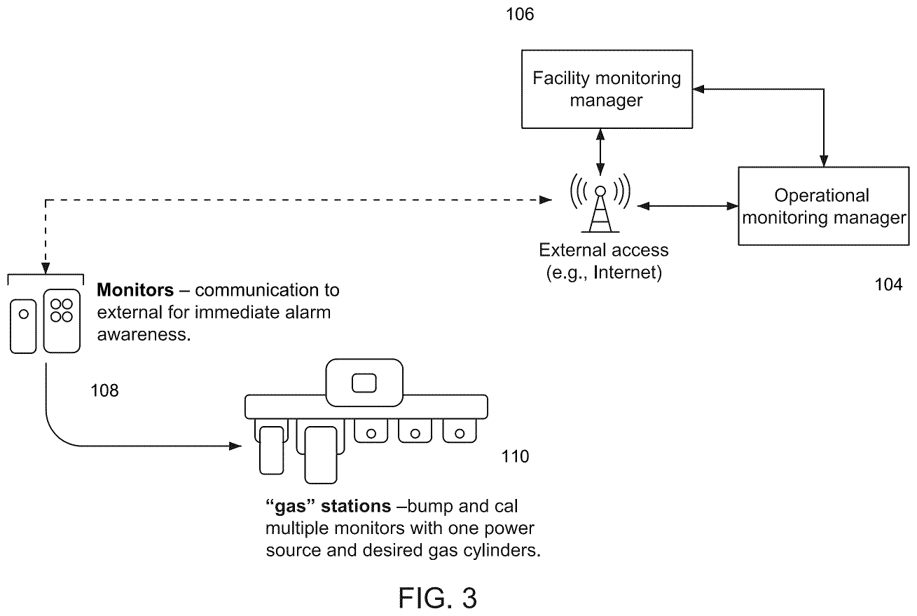

Referencing FIG. 3, it can be seen that a gas station 110 can reach external devices without a data manager, for example where one or more of the monitoring devices has sufficient capability to communicate externally (e.g., using cellular or satellite communications). In certain embodiments, the system of FIG. 3 can provide numerous benefits of the gas station as disclosed herein, without requiring the installation of a data manager in proximity to the gas station.

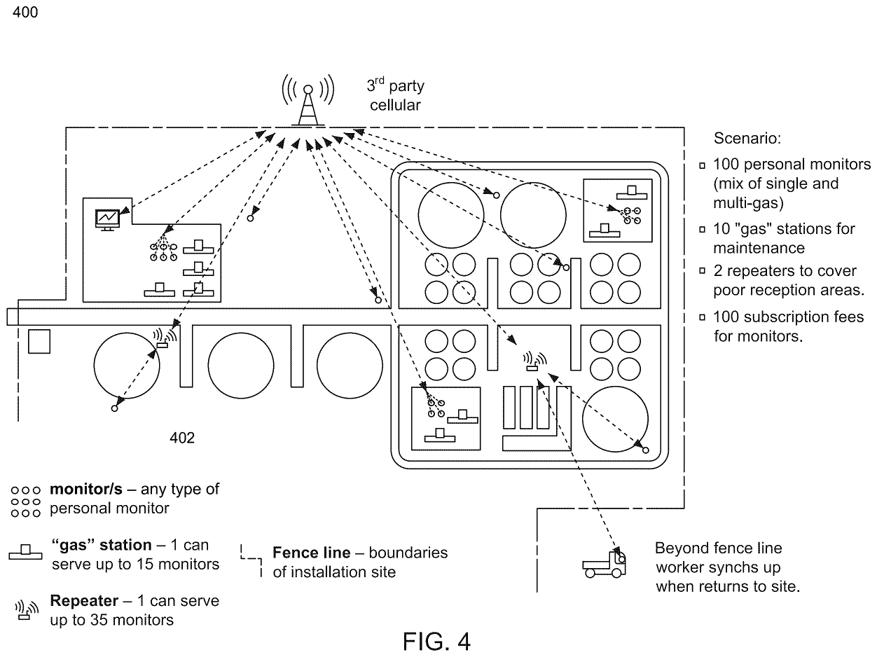

Referencing FIG. 4, another example gas monitoring system 400 is schematically depicted. In the example of FIG. 4, one or more monitoring devices in the system are capable of high power communications, for example utilizing cellular and/or satellite communications. The example of FIG. 4 utilizes repeaters 402 to provide connectivity in challenging areas, and to support gas stations. In some embodiments, the repeater 402 serves as a cellular booster. It should be understood that there is no limit to the number of monitors a repeater 402 can support. A system can be built with high capability monitoring devices as depicted in FIG. 4, and/or a system such as that depicted in FIG. 2 can be configured to utilize high capability monitoring devices as available within the system, while supporting low capability monitoring devices and the consequent benefits thereof (e.g., lower cost, reduced operational complexity, reduced power consumption). In some embodiments, one or more subscription fees are associated with the gas monitoring system or components thereof (e.g., on each monitor, on a group of monitors, on each gas station, on the system). The one or more subscription fees can appropriately attribute cost to the beneficiaries of the gas monitoring and/or provide an accounting lever to support efficient operation and/or iterative improvement of operations.

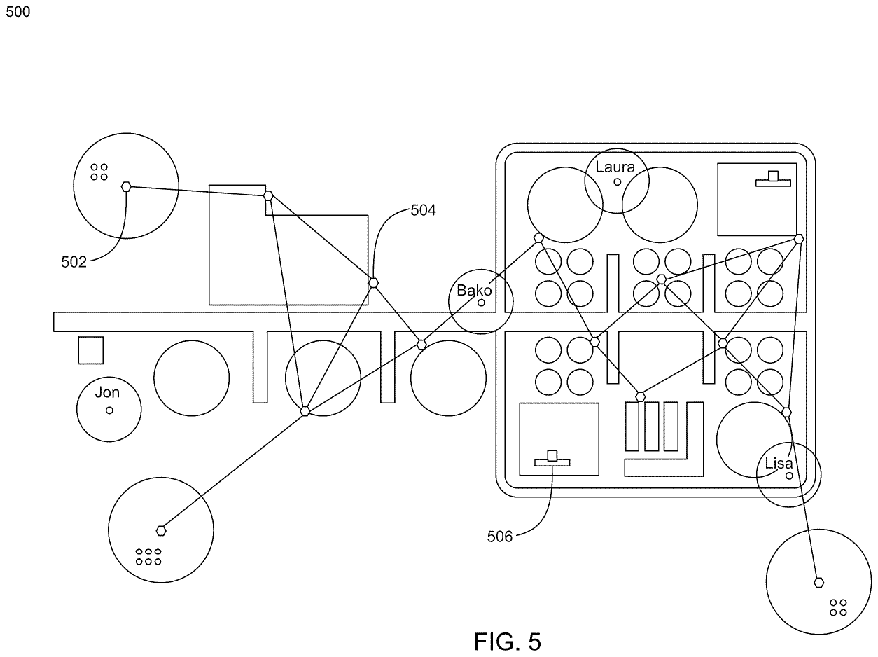

Referencing FIG. 5, another example gas monitoring system 500 is schematically depicted. The example of FIG. 5 includes the utilization of a number of low power anchors 504 that extend connectivity of the low power network as desired in the facility, including to create connectivity in challenging locations. The anchors form a static portion of a mesh network including the data managers 502, and the gas monitors, and optionally the gas stations 506, and have sufficiently low power requirements that a typical monitor in a convenient format can operate from battery for extended periods, up to about three years, without requiring a charge or battery change. In certain embodiments, one or more of the anchors 504 can be provided with one or more sensors, and can be used to rapidly provide a fence line or monitored region. For example, a notification or alarm may be generated, and optionally transmitted to other devices, if an instrument leaves a perimeter established by one or more of the data manager or an anchor. In certain embodiments, the anchors can be utilized to improve the available position information about specific gas monitors. In some embodiments, an operator or connected device location, such as a three-dimensional location (e.g., latitude, longitude, and height) may be determined from the placement of anchors in communication with operators/devices.

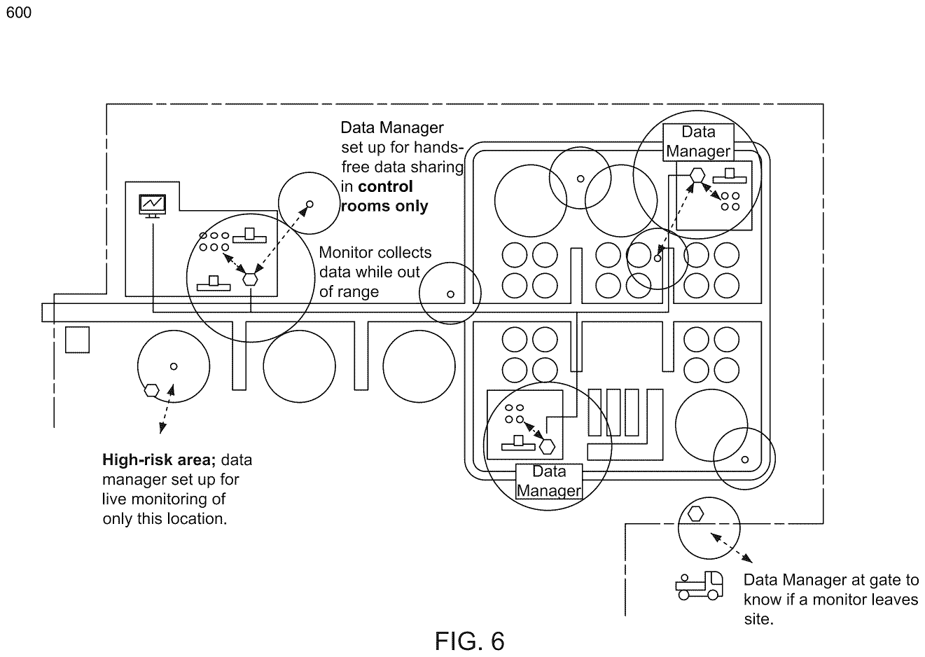

Referencing FIG. 6, another example gas monitoring system 600 is schematically depicted. The example of FIG. 6 includes a data manager positioned in a sensitive location, for example to ensure that connectivity in the area is successful, and to provide high rate data communication at the location—for example allowing for collection of faster sampling data, higher resolution data, keeping longer data sequences (e.g., a monitor out-of-connection or only connected through other low power network devices may be limited in the amount of data that can be buffered or saved), and/or supporting low latency analysis that may depend on a larger set of data than is typically provided by a monitor operating in an area that does not have constant high speed connectivity. In some embodiments of the example gas monitoring system 600, an anchor may be used in place of the data manager to provide connectivity and communication.

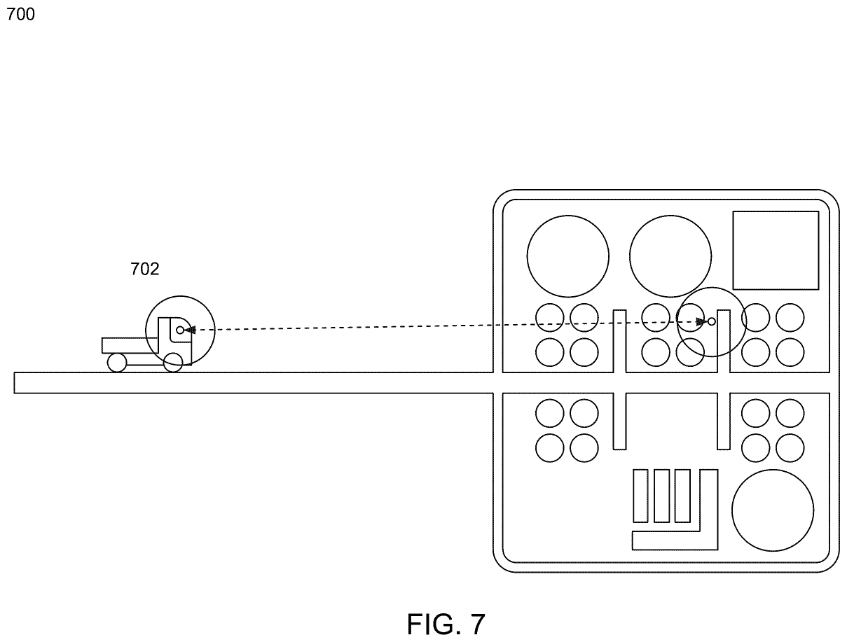

Referencing FIG. 7, an example gas monitoring system 700 is schematically depicted, with a monitoring device 702 currently depicted off location. In the example of FIG. 7, when the isolated monitoring device comes into communication range with a data manager, the data from the monitoring device is downloaded, and firmware and calibration data is confirmed and/or updated. In certain embodiments, the isolated monitoring device may also provide a notification to the user if a more involved operation is involved, for example if the firmware needs to be updated, the device requires a bump or calibration test, and/or if the device needs service or should be checked.

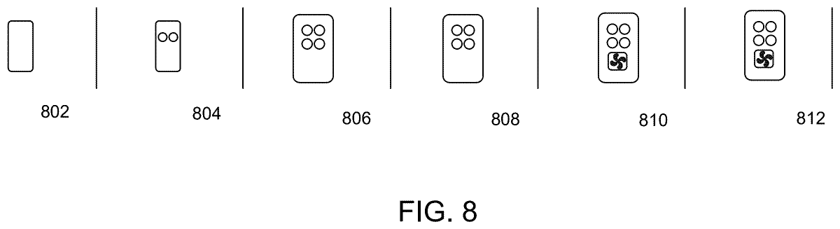

Referencing FIG. 8, a number of example monitoring devices are schematically depicted. Devices can range from a single gas sensing device to a multi-gas sensing device, and can include passive or active sensors (e.g., forced flow sensors). In certain embodiments, monitoring devices may include other types of sensors as indicated preceding. In certain embodiments, expensive or complex gas monitoring devices may also further include higher power network connectivity options (e.g., cellular and/or satellite), given the lower cost ratio of providing such connectivity in view of the cost of the device overall. However, even where expensive or complex gas monitoring devices are present, those devices may nevertheless not include higher power network connectivity (e.g., for security reasons, and/or to minimize power consumption), and/or the high power communications may be reduced where possible to reduce power consumption, and accordingly such devices provide additional capability in certain embodiments, and still benefit from a gas monitoring system having multiple device classes. Monitor 802 does not detect gas, gas monitor 804 detects a single gas, gas monitor 806 is a portable diffusion monitor configured to detect up to 4 gases, gas monitor 808 is a portable diffusion monitor configured to detect up to 5 gases, including exotic gases, gas monitor 810 is an aspirated gas monitor configured to detect up to 4 gases, and gas monitor 812 is an aspirated gas monitor configured to detect exotic gases.

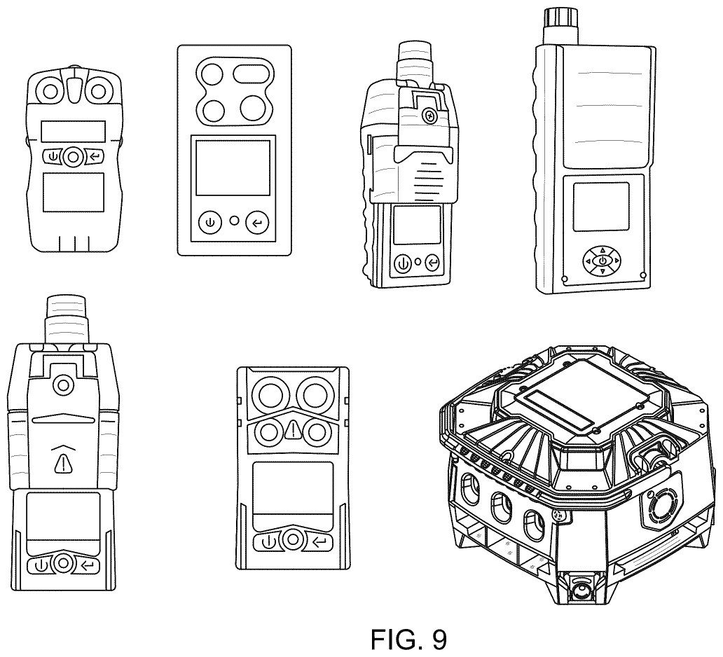

Referencing FIG. 9, example and non-limiting gas monitoring devices compatible with systems herein are schematically depicted. Depending upon the system configuration and the current operations, such devices may operate as a monitoring device, as an anchor, and/or as a data manager.

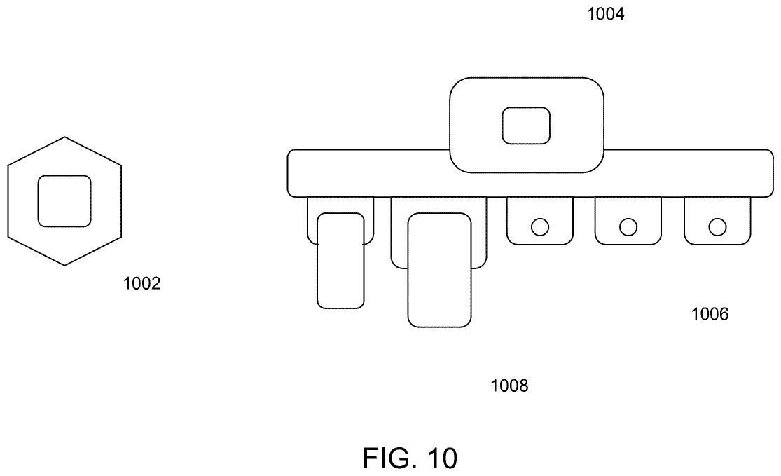

Referencing FIG. 10, an example data manager component 1002 (left) is schematically depicted, and shown as in the preceding figures. FIG. 10 further depicts a gas station component 1004, having two different monitor components 1008 docked thereon at gas monitor physical interfaces 1006, depicted as shown in the preceding figures. The example physical interfaces 1006 allow the gas station to provide power to the docked gas monitor, to communicate with the gas monitor (e.g., to download data from the gas monitor, to perform diagnostics and/or retrieve diagnostic data, to perform calibration operations, and/or to update firmware and/or calibrations of the gas monitor), and/or to provide gas flow to the gas monitor (e.g., providing a selected gas to a sensing element of the gas monitor, for example to perform a bump test and/or a response characterization of the sensing element to a selected gas at a selected concentration).

In certain embodiments, the data manager class component manages firmware for the monitoring device class components. In certain embodiments, the data manager component interprets data from the monitoring device in response to planned conditions, for example the monitoring device entering proximity with the data manager and/or the monitoring device engaging with a dock on a gas station class component, and determines that the firmware on the monitoring device should be updated (e.g., checking versions or other indicators for the firmware on the device), and in response to determining the firmware should be updated performs operations to update the firmware. In certain embodiments, the firmware may be updated during operations where the monitoring device is known to be in a stable place and will not be relied upon for monitoring operations, for example during a dock on a gas station, while in a storage location, and/or during a time of day when the associated operator for the gas monitor (if applicable) does not work, or typically does not work. Additionally or alternatively, the firmware update may include providing a notification from the monitor (e.g., an alert, light, displayed message, selected sound, etc.) that monitoring operations are temporarily unavailable during firmware update operations. In certain embodiments, for example in systems where the monitors do not have predictable downtime periods, and/or where it is undesirable that monitors have a downtime period, the data class manager component can download the new firmware in chunks, for example providing chunk sizes that are reasonable to download from the data manager during transient communication operations (e.g., within 10 seconds) between the data manager and the gas monitoring device. Each chunk can be downloaded and confirmed, or repeated as needed, until all of the chunks of the firmware update (or a portion of the firmware that can be implemented) are provided, and then later at a selected time the firmware update can be implemented. The final implementation of the firmware update can be commanded by the data manager directly (e.g., determining to flip the switch during a docking operation, expected downtime, when the monitor is in a selected location such as storage, etc.), or by a controller on the gas monitor, for example responding to a flag set by the data manager than a firmware update should be performed. In certain embodiments, the controller on the gas monitor can provide various feedback related to the firmware update, for example confirming the receipt of firmware download chunks, confirming the update of the firmware, and/or providing any messages or logs related to errors, failure to complete the update, and/or improper operation after the update.

In certain embodiments, the gas station includes a controller configured to perform various operations of the gas station, including for example performing bump tests, calibration operations, communicating with the data manager (e.g., to receive updated firmware, to confirm bump/cal data, etc.). In certain embodiments, the resulting data from the bump/cal operations is passed to the data manager from the gas station, and/or the data from the bump/cal operations may additionally be stored on the respective gas monitoring device and/or communicated to the data manager from the respective gas monitoring device. In certain embodiments, the controller on the gas station includes controlling gas flow from one or more test gases (e.g., controlling flow routing between various gas bottles and the docked monitor(s)) to docked gas monitor(s) to perform bump/cal operations, reporting on gas bottle levels, and/or providing information about faults, diagnostic operations, or the like related to the gas delivery system, docking and communication system, and/or related to charging operations (where applicable).

In certain embodiments, the interaction of the high power network connection to external devices, combined with a low power mesh network of devices on a gas monitoring system, combine to allow for a number of operations described following. An example operation includes a situational interaction operation with specific gas monitor(s) based on operating conditions within the facility. For example, alarm thresholds within the gas monitor may be adjusted according to the operating conditions. In a further example, a person performing a specific action that is visible to the system, for example according to specific procedures such as the completion of forms (e.g., a form completed for a confined space entry, a lockout/tagout operation, or the like, which may be electronically submitted and have fields therefore that can be interpreted by a controller on the data manager, by the facility monitoring manager, by the operational monitoring manager, and/or by the individual gas monitor for that operator.

In response to the operating conditions, the appropriate controller(s) may be configured to perform a situation interaction operation such as: commencing or stopping monitoring operations; adjusting an alarm threshold value (e.g., move from 5 ppm alarm to 25 ppm alarm; moving from a 1-second average based determination to a 3-second average based determination; etc.); adjusting an alarm response value (e.g., changing a location of alarm communications, changing alarm content such as selected lights, sounds, and/or messages, etc.); and/or adjusting a secondary parameter related to the gas monitoring system, such as an evacuation routing, adjusting a distance value for relating monitors at the facility (e.g., a system that utilizes 25 feet to consider those monitors to be related during nominal operations, switches to 10 feet to consider monitors to be related during high pressure operations on a tank having a highly toxic gas; and/or could include switching a facility relationship map from a first geofencing regime to a second geofencing regime—for example to change the risk assessment of the entire facility based on a global parameter such as “producing product A”, “producing product B”, “during Alert Type A”, “inclement weather preparation”, etc.). Without limitation to any other aspect of the present disclosure, example and non-limiting situational interaction operations include one or more operations such as: adjusting an allowable power consumption value for a device in the gas monitoring system; adjusting a monitoring operation; adjusting an alerting operation; adjusting an alert response operation; providing a selected notification to a selected external device; setting a flag to command a calibration and/or firmware update to a device (e.g., which may be used by a controller on the gas station and/or the data manager on a next docking event for the gas monitor); and/or setting a flag for a device to be turned in for analysis (e.g., which may be used anywhere in the system, for example the gas station may avoid performing bump/cal operations on such a device to avoid forensic complications, and/or the device may give the user a notice to turn it in to service or maintenance, etc.).

In certain embodiments, operations herein are performed to determine an operating condition for the facility, for a portion of the facility (e.g., an absolute portion such as “near the distillation column” or a relative portion such as “within 30 feet of gas monitor XYZ”), for a gas monitor, and/or for an operator associated with a gas monitor. Such operations may be performed by a controller (or controllers) in the system, including a controller positioned (at least in part) on the operational monitoring manager, the facility monitoring manager, a data manager, an anchor, a gas monitor, and/or the gas station. In certain embodiments, the respective operating condition is determined directly from data available to the system (e.g., users may check in and out to the facility, providing an electronic indication of whether they are present; the facility monitoring manager may provide information to the operational monitoring manager, such as a global operating state of the facility (e.g., RUN, SHUTDOWN, PROCESS A, STAGE B, etc.), one or more operating parameters for the facility and/or equipment thereof, or the like. In certain embodiments, the respective operating condition may be inferred and/or estimated based on other parameters (e.g., the calendar, weather events, number of personnel at the facility, etc.), and/or the respective operating condition may be explicitly set by and administrator or supervisor, and/or directly available system information such as information parsed from procedural data such as entry of a lockout/tagout procedure that is available to the facility monitoring manager or another controller in the system. In certain embodiments, the respective operating condition may be inferred from location of personnel, utilization of sensors to detect personnel conditions (e.g., the wearing of selected personal protective equipment, which may be determined based on utilization of smart equipment such as a smart respirator mask, and/or inferred from camera information). In certain embodiments, the type of operation, location of personnel, the hazards to be monitored, and the PPE worn by the operator, may all be utilized to determine the current threshold levels for alerts and alert responsive activity.

In certain embodiments, the gas monitoring system allows for specific messages and/or messaging/alerting/notification regimes to be provided in response to selected instruments within the gas monitoring system. Such messages can be sourced from any device in the system, for example from the facility monitoring manager, the operational monitoring manager, and/or one or more of the gas monitors, and can be provided to any device or selected group of devices within the gas monitoring system. For example, a message can be provided to all gas monitors in a particular area of the facility, within a certain distance from another gas monitor (e.g., a gas monitor that has an issue or an active alert), to a defined list of gas monitors (e.g., associated with a certain personnel group, regardless of location), to a defined list of devices, and/or to a contingent list of devices (e.g., all gas monitors that have been calibrated within the last 10 days). The messages can vary in priority and/or importance, with a selectively scheduled delivery such sending a message the next time each gas monitor enters a data manager zone, and/or can be utilized to override normal behavior—for example allowing a first high priority message to be propagated to all available devices right away (e.g., chaining on the low power network), and only providing a second lower priority message to be propagated to devices under non-disruptive conditions (e.g., to preserve power utilization for devices where the message is not urgent). In certain embodiments, a gas monitor user can send a message from their gas monitor to other gas monitors in the area, to the facility monitoring manager, and/or to the operational monitoring manager.

In certain embodiments, a high capability device, such as a gas monitoring device having a cellular and/or satellite communication option, may be utilized to bridge network portions within the gas monitoring system, for example to support gas monitoring devices and/or gas stations in an area of the facility where a full data manager is not desired, or not available. The extension of the network using a high capability bridging device allows for operations of the network to be supported beyond the original installation limits, for example to respond to temporary and/or highly transient changes in the gas monitoring environment for the facility, without waiting for or requiring a full installation using an anchor or data manager.

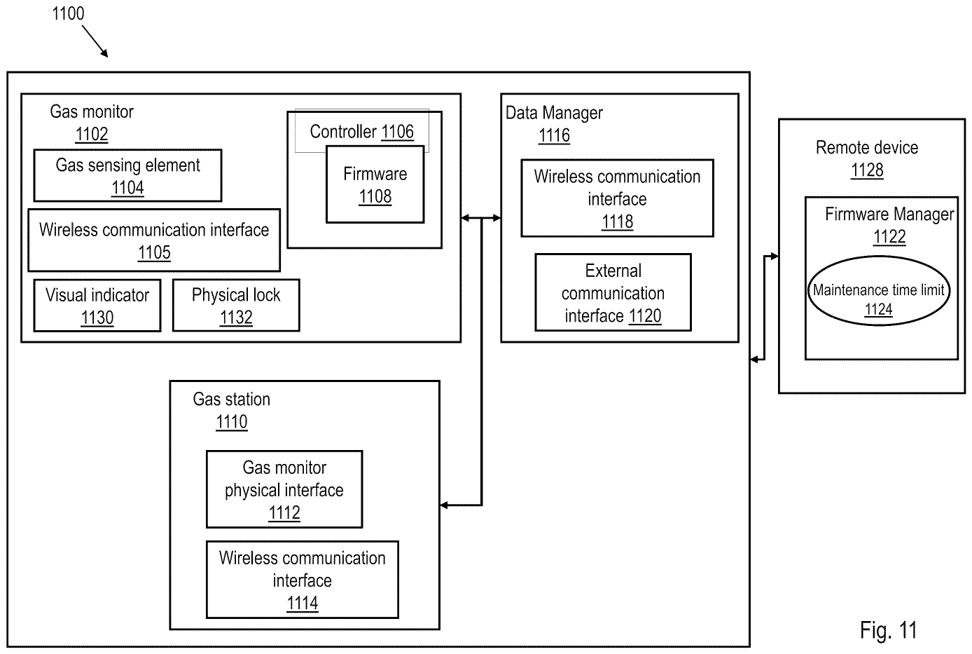

Referring now to FIG. 11, an example system 1100 includes operations for firmware execution for instruments in a monitoring system, including operations to chunk firmware downloads and implement firmware updates at an appropriate time. The system 1100 for gas monitoring includes a gas monitor 1102 including a gas sensing element 1104, a wireless communication interface 1105, and a controller 1106 having firmware 1108 installed thereon, the firmware 1108 including instructions executable by the controller 1106 to perform operations of the gas monitor 1102. The instructions could be code, algorithms, calibrations, applications, application add-ins, an operating system, operating system support, or the like. Operations can include at least the sensing or communication functions. The controller 1106 should be understood in the broadest sense, not just as a processor, but should be understood to include processing, memory, and/or communication resources.

The example system 1100 also includes a gas station 1110 including at least one gas monitor physical interface 1112, and a wireless communication interface 1114. The example system 1100 also includes a data manager 1116 including a wireless communication interface 1118 and an external communication interface 1120. The example system 1100 also includes a firmware manager 1122 configured to apply the firmware 1108 for the gas monitor 1102 in response to the gas monitor 1102 coupling to the at least one gas monitor physical interface 1112, wherein the operations to apply the firmware 1108 are performed within a maintenance time limit 1124. Applying the firmware includes either installing or updating the firmware. In some embodiments, the data manager 1116 is configured to provide firmware updates to the gas station 1110. In some embodiments, the firmware manager 1122 is configured to provide firmware updates to the gas station 1110. Firmware updates to the gas station 1110 can be executed wirelessly, and in some embodiments, are provided through an anchor.

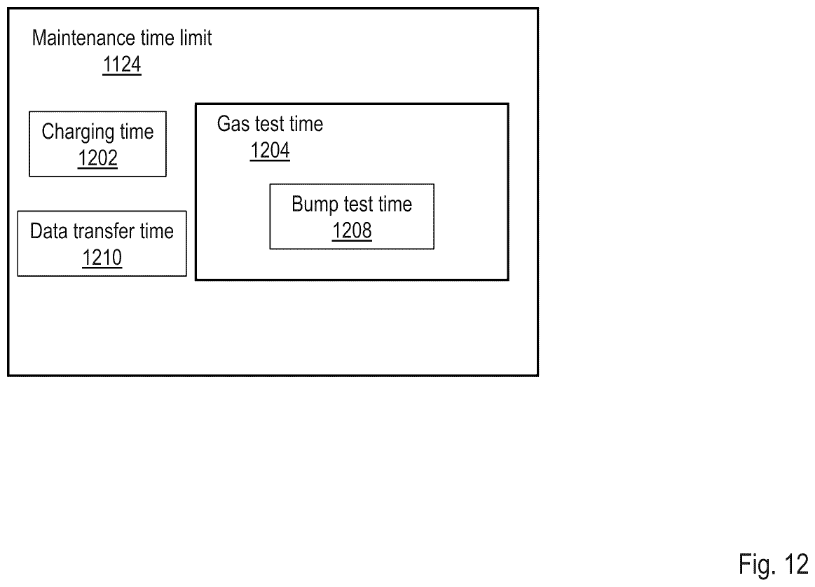

The maintenance time limit 1124 refers to the time that the gas monitor 1102 will inherently be coupled to the gas station 1110, which is within the time utilized to perform charging, data transfer (as applicable), and/or bump testing (e.g., operations to test the responsiveness of the sensor element of the gas monitor to either the target detected gas and/or a test gas with a known correlated response). By limiting the operations to apply the firmware 1108 to a maintenance time limit 1124, firmware updates do not add any incremental time or inconvenience to the operator or system. For example, and with reference to FIG. 12, the maintenance time limit 1124 includes one or more of a charging time 1202, a gas test time 1204 (e.g., bump test time 1208, and/or may include a more comprehensive gas test such as a full response characterization operation of the sensing element), and/or comprises a data transfer time 1210 for the gas monitor (e.g., where the gas monitor transfers data while coupled to the gas station, for example to preserve power utilization of the gas monitor; the gas monitor may additionally or alternatively perform data transfer operations while communicatively coupled to the data manager and/or an anchor of the system, and/or may transfer data through the mesh network, for example using other gas monitors, for certain types of data transfer and/or for certain operating conditions). Gas test time 1204 could refer to time associated with a full calibration or characterization of the sensor, other tests or diagnostics (e.g., due to a fault code, event such as high gas detection, and/or periodically), or it can refer to bump test time 1208. Data transfer in the system 1100 can occur through the mesh network, or directly to the data manager 1116 when in range. However, there may be some data dump/transfer performed when docking of the gas monitor 1102 to the gas station 1110 occurs, which could be normal or done only under some circumstances.

While FIG. 11 depicts the firmware manager as residing on a separate logical device (e.g., remote device 1128), it should be understood that it can be distributed to, at least in part, or positioned on, at least in part, any or all of the controller 1106, the data manager 1116, the gas station 1110, or the remote device 1128. For example, components of the firmware may be distributed among the components of the system 1100.

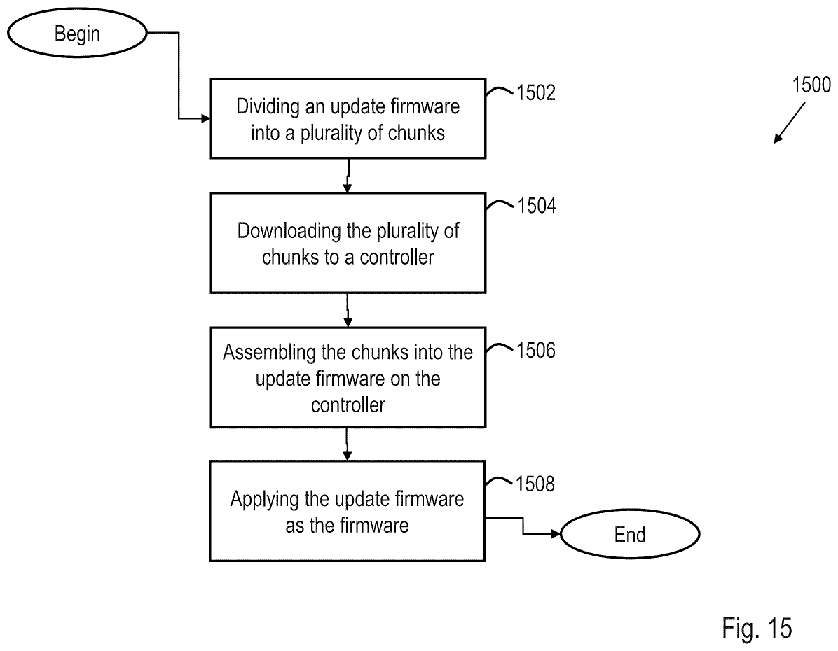

In embodiments of the system 1100, coupling to the at least one gas monitor physical interface 1112 includes a docking event, and the firmware manager 1122 is further configured to apply the firmware 1108 over a number of docking events. In this way, the operation of applying firmware does not cause burden on the system or operator. The firmware manager 1122 is further configured to apply the firmware 1108 by performing the following operations, as depicted in the procedure 1500 shown in FIG. 15. The procedure 1500 includes an operation 1502 of dividing an update firmware into a plurality of chunks (e.g., 100 kb chunks, 1 MB chunks, etc.), an operation 1504 of downloading the plurality of chunks to the controller, an operation 1506 of assembling the chunks into the update firmware on the controller, and an operation 1508 of applying the update firmware as the firmware. The operation 1506 of assembling and the operation 1508 of applying can optionally be performed while undocked. Optionally, the final firmware install may only be done while the gas monitor is docked (which can include determining the estimated docking time to ensure the final install can be completed before docking operations are done.).

In some embodiments, the firmware manager 1122 is further configured to estimate the maintenance time limit 1124 for a specific docking event, and to apply the firmware 1108 in response to the estimated maintenance time limit event. In these embodiments, the firmware manager 1122 is further configured to divide the update firmware into the plurality of chunks in response to the estimated maintenance time limit event.

In some embodiments, the controller 1106 is configured to command a visual indicator 1130 of the gas monitor 1104 during the operations to apply the firmware 1108, for example with a light or display indicator on the gas monitor and/or the gas station indicating that sensitive operations are in process and the gas monitor should not be disturbed. In certain embodiments, operations to apply the firmware 1108, for example to install and/or utilize an update to the firmware, may be performed during docking operations with the gas monitor, and/or may be performed at another time, for example during downtime when the gas monitor is not directly in use for sensing operations, after all segments of the firmware are downloaded to the gas monitor. In some embodiments, the firmware manager is configured to command a physical lock 1132 of the gas monitor (e.g., a physical engagement during docking that keeps it from being unplugged) during at least a portion of the operations to apply the firmware 1108. The physical lock 1132 would enforce docking during firmware operations, and where used the physical lock 1132 provides a more certain confirmation that the gas monitor will not be disturbed during sensitive operations than only the visual indicator. In embodiments, some operations performed off-dock would not apply (e.g., if installation occurs off-dock).

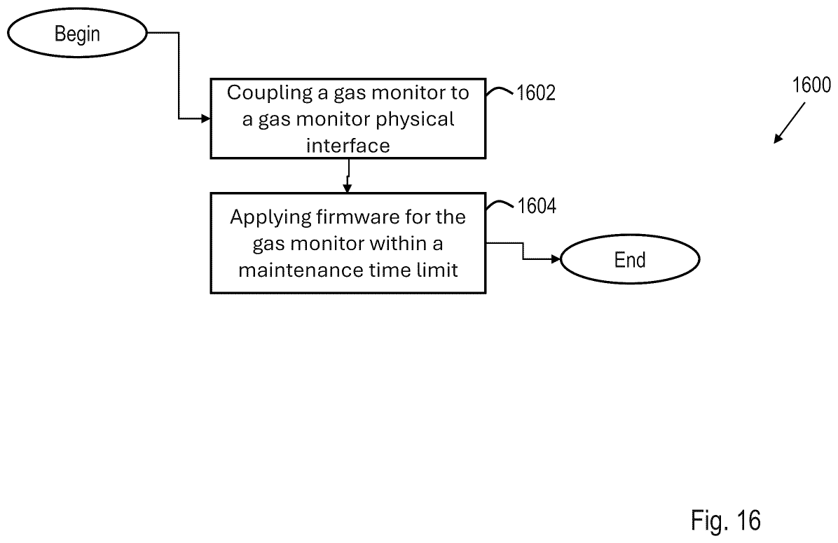

The example system 1100 is configured to perform the following operations, as depicted in the procedure 1600 shown in FIG. 16: an operation 1602 of coupling a gas monitor to a gas monitor physical interface and an operation 1604 of applying firmware for the gas monitor within a maintenance time limit. In some embodiments, an operation may include applying the firmware over a number of docking events. This operation may further include estimating the maintenance time limit for a specific docking event, applying the firmware in response to the estimated maintenance time limit event, and dividing the update firmware into a plurality of chunks in response to the estimated maintenance time limit event. The example system 1100 is configured to also perform an operation of commanding a visual indicator of the gas monitor during the operations to apply the firmware or commanding a physical lock of the gas monitor during at least a portion of the operations to apply the firmware.

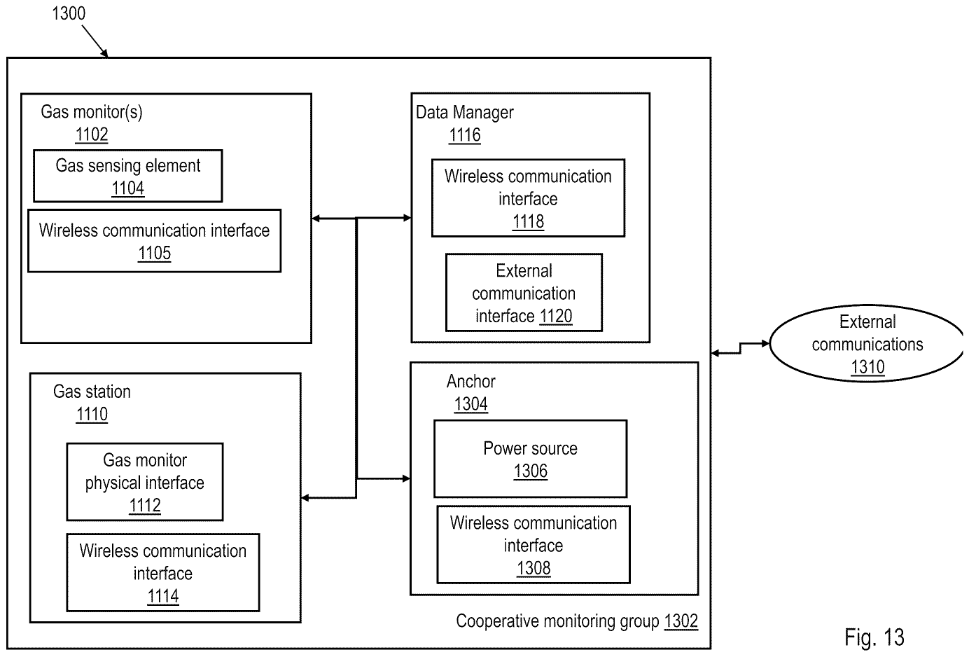

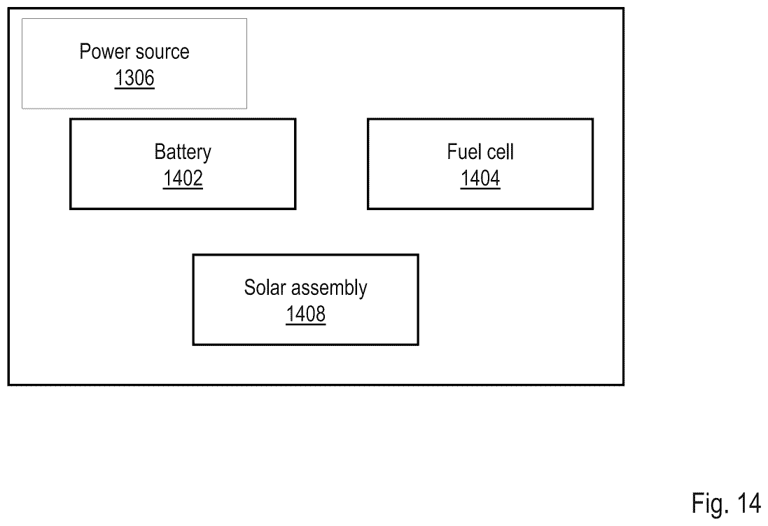

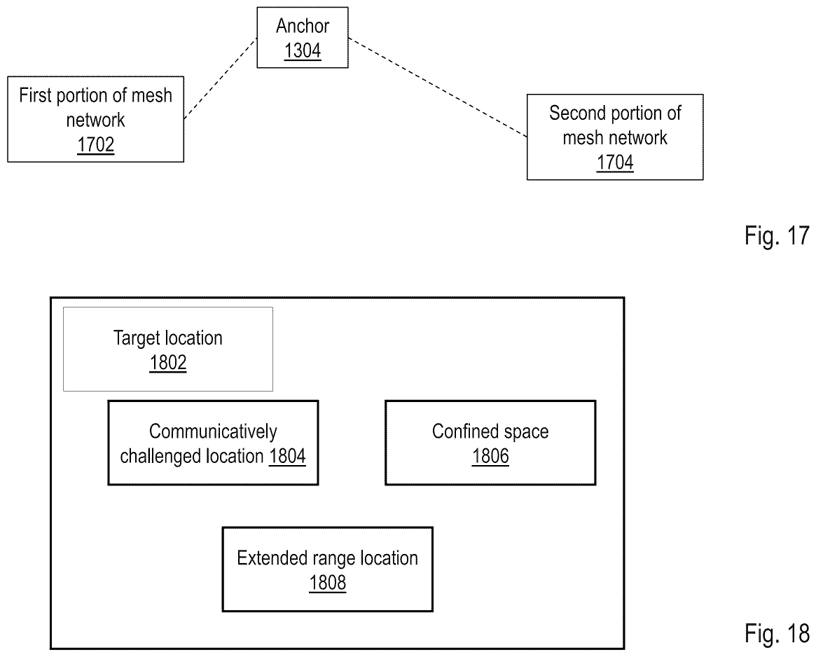

Referring now to FIG. 13, an example system 1300 includes unpowered anchors for range extension. In some embodiments, anchors 1304 are able to operate for a long duration on battery (e.g., 3 years), can be integrated into a network, can serve as a beacon or fence line member, can perform condition tracking, and the like. The system 1300 for gas monitoring can include a cooperative monitoring group 1302 for gas monitoring. The cooperative monitoring group 1302 includes a plurality of gas monitors 1102 including a gas sensing element 1104 and a wireless communication interface 1105. The cooperative monitoring group 1302 also includes a gas station 1110 including at least one gas monitor physical interface 1112, and a wireless communication interface 1114. The cooperative monitoring group 1302 also includes an anchor 1304 including an independent power source 1306 and a wireless communication interface 1308. For example, and referring to FIG. 14, the power source 1306 can be at least one of a battery 1402, fuel cell 1404, or a solar assembly 1408. The cooperative monitoring group 1302 also includes a data manager 1116 including a wireless communication interface 1118 and an external communication interface 1120. The cooperative monitoring group 1302 is configured to operate as a mesh network. External communications 1310 of the cooperative monitoring group 1302 from the plurality of gas monitors 1102, the gas station 1110, and the anchor 1304, are routed through the data manager 1116. With reference to FIG. 17, the anchor 1304 bridges a first portion of the mesh network 1702 with a second portion of the mesh network 1704. The first portion of the mesh network 1702 may be considered to be the main network on one side (e.g., first portion), and extended range monitors on the other side (e.g., second portion). It should be understood that bridging could be intermittent, such as when the data monitors are not in direct contact with a data manager 1116. In some embodiments, the anchor 1304 is communicatively coupled to at least one gas monitor 1102, and the anchor serves 1304 as a data manager link for at least one of the plurality of gas monitors 1102. In these embodiments, communications from this gas monitor are confined to be transmitted through the anchor 1304.

Continuing with reference to FIG. 13, the anchor 1304 comprises a fixed position. In some embodiments, and with reference to FIG. 18, the anchor 1304 is positioned in communication proximity to a target location 1802, such as a communicatively challenged location 1804, a confined space 1806, or an extended range location 1808. In some embodiments, the anchor 1304 is a low infrastructure anchor, which means it may have no power connection and/or no communications connection (e.g., wired and/or line-of-sight comms).

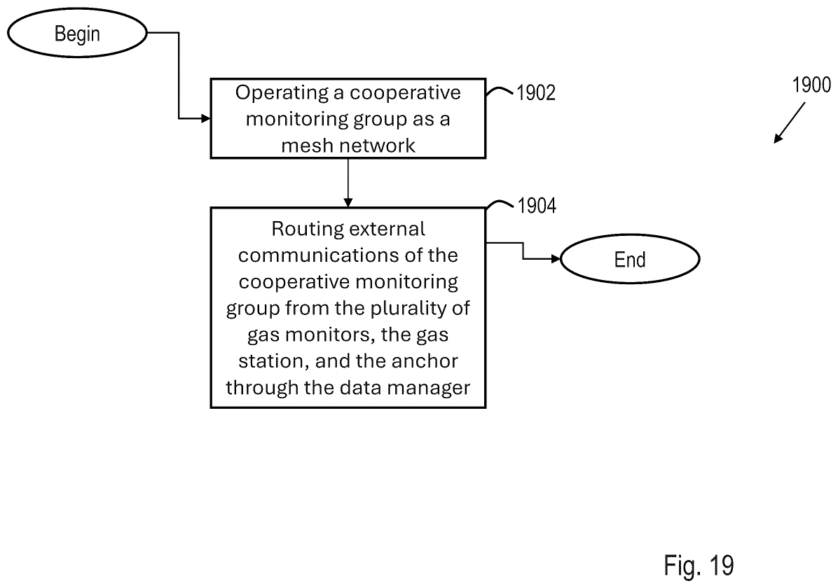

The example system 1300 is configured to performing the following operations, as depicted in the procedure 1900 shown in FIG. 19: an operation 1902 of operating a cooperative monitoring group as a mesh network, and an operation 1904 of routing external communications of the cooperative monitoring group from the plurality of gas monitors, the gas station, and the anchor through the data manager.

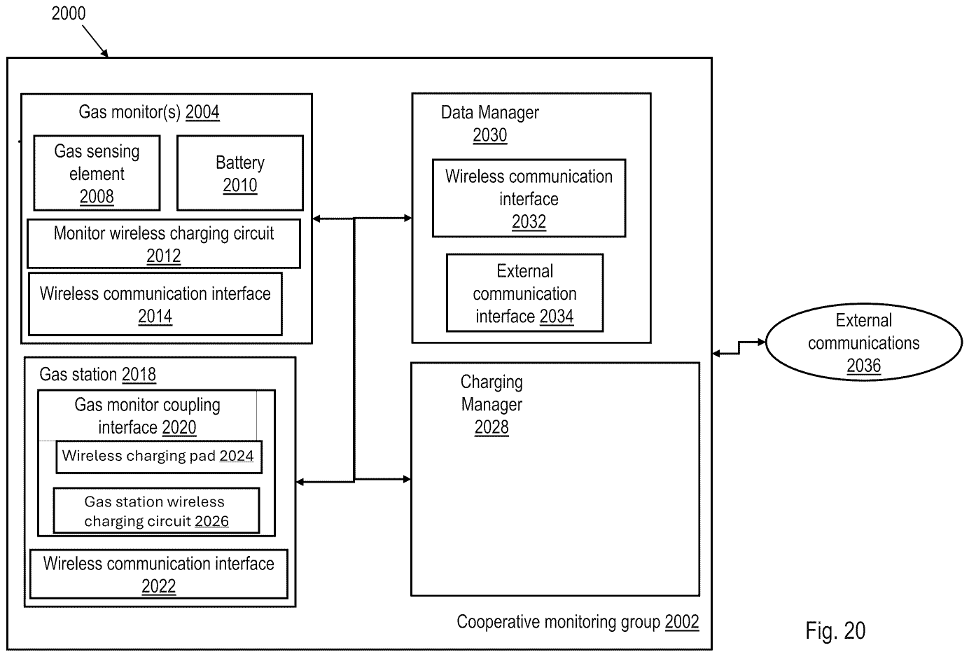

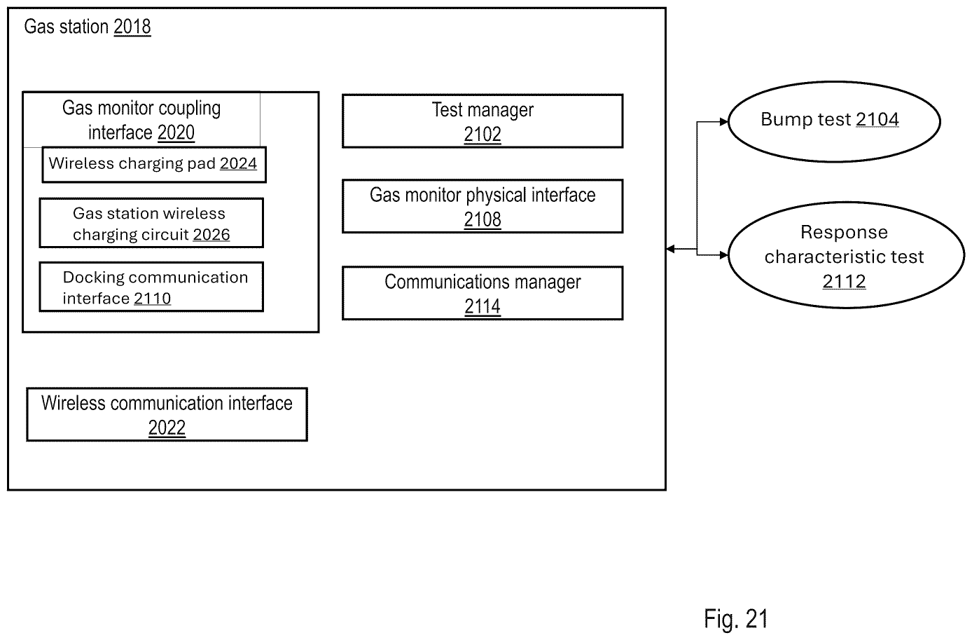

Referring now to FIG. 20, a system 2000 for gas monitoring includes a cooperative monitoring group 2002 that is capable of supporting wireless instrument charging. The cooperative monitoring group 2002 includes a plurality of gas monitors 2004 comprising a gas sensing element 2008, a battery 2010, a monitor wireless charging circuit 2012, and a wireless communication interface 2014. The cooperative monitoring group 2002 includes a gas station 2018 including at least one gas monitor coupling interface 2020 and a wireless communication interface 2022. The at least one gas monitor coupling interface 2020 includes a wireless charging pad 2024 and a gas station wireless charging circuit 2026. The cooperative monitoring group 2002 also includes a charging manager 2028 configured to operate the gas station wireless charging circuit 2026 to charge the battery 2010 of an associated gas monitor 2004 in response to the associated gas monitor 2004 positioned in proximity to the wireless charging pad 2024. The cooperative monitoring group 2002 also includes a data manager 2030 including a wireless communication interface 2032 and an external communication interface 2034. The cooperative monitoring group 2002 is configured to operate as a mesh network, and external communications 2036 of the cooperative monitoring group 2002 from the plurality of gas monitors 2004 and the gas station are 2018 routed through the data manager 2030.

In embodiments, the gas station further comprises at least one gas monitor physical interface 2108 configured to expose the gas sensing element 2008 to a selected gas constituent. In embodiments, the gas station 2018 further includes a test manager 2102 configured to perform a bump test 2104 of the gas sensing element 2008 and/or perform a response characteristic test 2112 of the gas sensing element 2008.

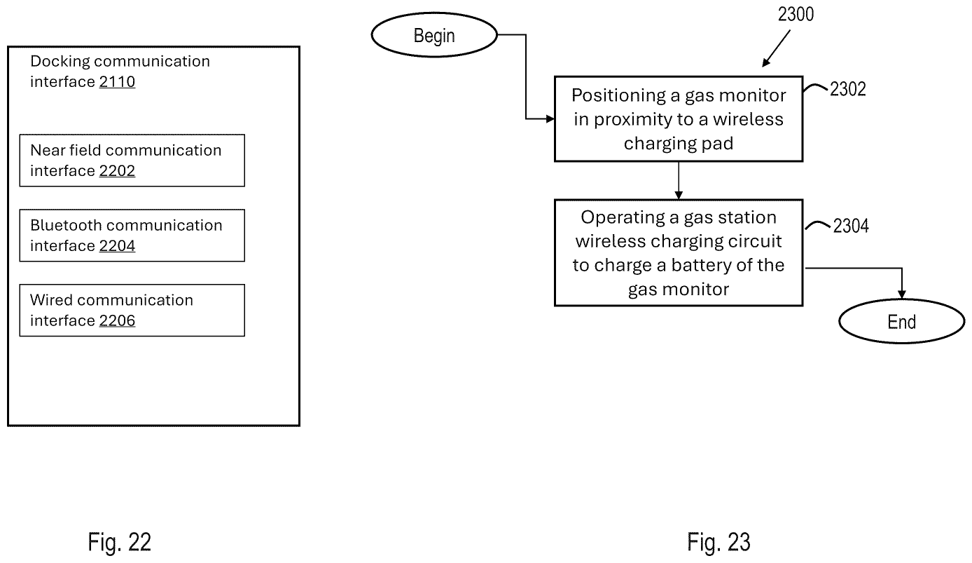

In embodiments, the gas monitor coupling interface 2020 includes a docking communication interface 2110, which can be a near field communication interface 2202, a Bluetooth communication interface 2204, or a wired communication interface 2206, as seen in FIG. 22. When a gas monitor is coupled to a gas station, communication interface can be changed such as for example to use a lower power and/or more reliable communication interface. A lower power interface may be desired if the gas monitor is close and in a known location when docked (e.g., for non-wired) or if it is wired. A more reliable interface may be desired during charging operations since power consumption is a low concern. Having access to a more reliable communications interface enables data transfer that is more reliable (e.g., higher power and/or steered communications) and/or higher rate.

In embodiments, the gas station further comprises a communication manager 2114 configured to modulate the gas station wireless charging circuit 2026 to provide communications between a coupled gas monitor 2004 of the plurality of gas monitors, and the gas station 2018.

In embodiments, the monitor wireless charging circuit 2012 and the gas station wireless charging circuit 2026 are configured to transmit power from the gas station 2018 to a coupled gas monitor 2004 of the plurality of gas monitors using an inductive coupling and/or a photonic coupling (e.g., including resonance coupling, laser/coherent power transmission, or radio power transmission).

The example system 2000 is configured to performing the following operations, as depicted in the procedure 2300 shown in FIG. 23: an operation 2302 of positioning a gas monitor in proximity to a wireless charging pad, and an operation 2304 of operating a gas station wireless charging circuit to charge a battery of the gas monitor.

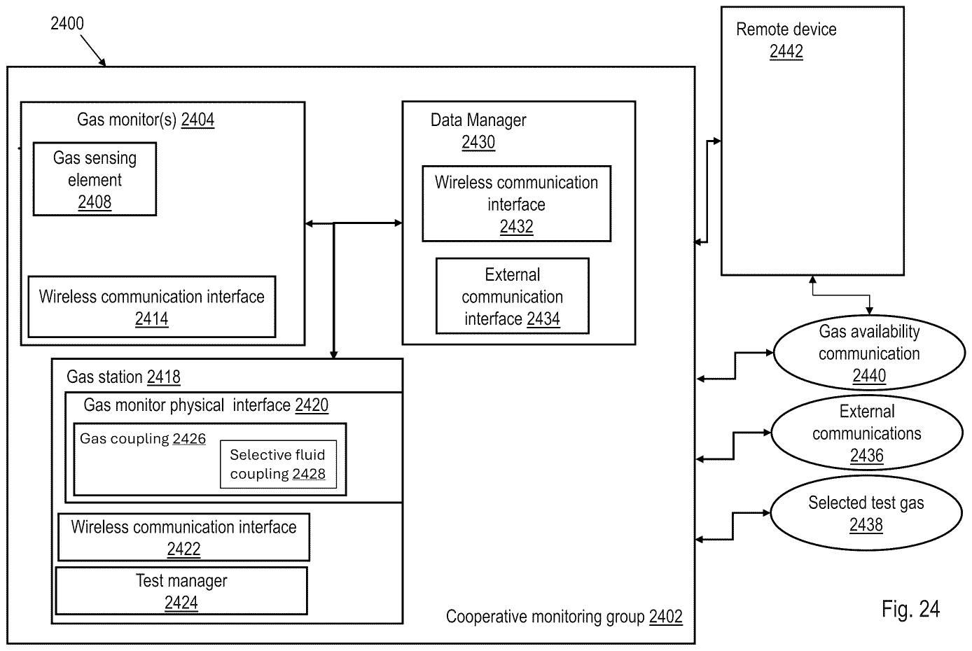

Referring now to FIG. 24, an example system 2400 for gas monitoring includes a cooperative monitoring group 2402 that is enabled to perform improved bump testing as well as other diagnostic, calibration, or audit tests, in some embodiments. The cooperative monitoring group 2402 includes a plurality of gas monitors 2404 comprising a gas sensing element 2408 and a wireless communication interface 2414. The cooperative monitoring group 2402 also includes a gas station 2418 including at least one gas monitor physical interface 2420 comprising a gas coupling 2426, a wireless communication interface 2422, and a test manager 2424 configured to perform a sensing element response test (e.g., response characteristic test) of the gas sensing element 2408. The cooperative monitoring group 2402 also includes a data manager 2430 including a wireless communication interface 2432 and an external communication interface 2434. The cooperative monitoring group 2402 is configured to operate as a mesh network, and external communications 2436 of the cooperative monitoring group 2402 from the plurality of gas monitors 2404 and the gas station 2418 are routed through the data manager 2430. In some embodiments, the sensing element response test is a bump test.

In some embodiments, the gas coupling 2426 is a selective fluid coupling 2428 between the gas sensing element 2408 and a selected test gas 2438. The test manager 2424 is further configured to provide a gas availability communication 2440 for the selected test gas 2438 to a remote device 2442. The test manager 2424 is further configured to sequentially perform a plurality of sensing element response tests for a gas monitor 2404, of the plurality of gas monitors, having a plurality of gas sensing elements.

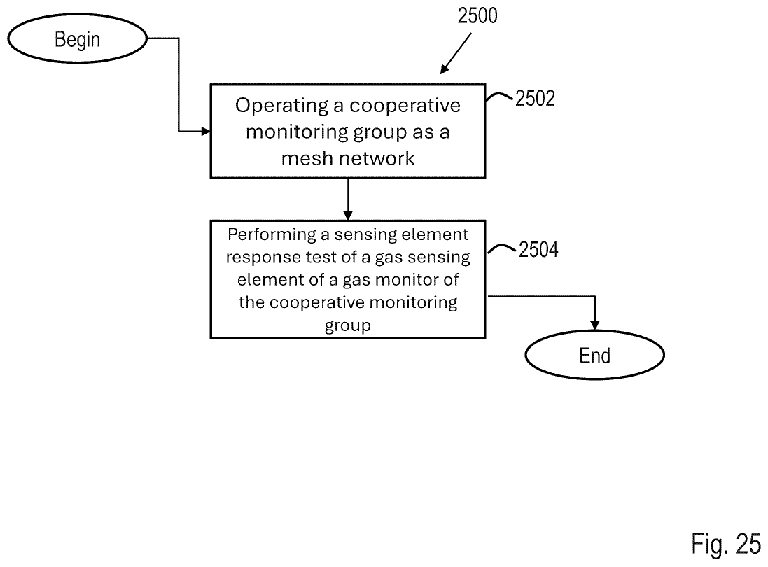

The example system 2400 is configured to perform the following operations, as depicted in the procedure 2500 shown in FIG. 25: an operation 2502 of operating a cooperative monitoring group as a mesh network, and an operation 2504 of performing a sensing element response test of a gas sensing element of a gas monitor of the cooperative monitoring group. Operations also include providing a selective fluid coupling between the gas sensing element and a selected test gas, providing a gas availability communication for the selected test gas to a remote device, sequentially performing a plurality of sensing element response tests for a gas monitor, and routing external communications of the cooperative monitoring group from the plurality of gas monitors, the gas station, and the anchor through the data manager.

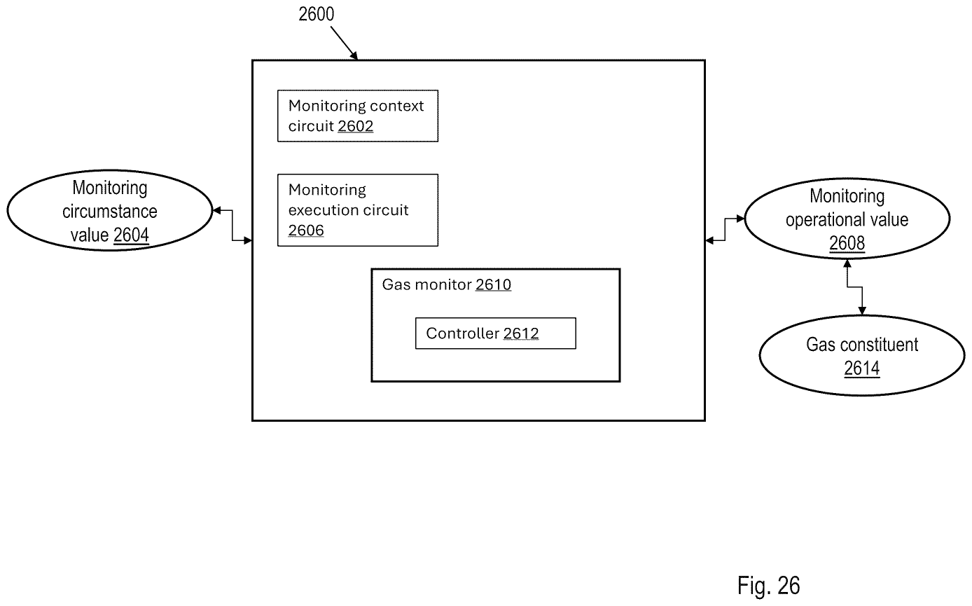

Referring now to FIG. 26, an example system 2600 is configured to enable and/or address situational interaction, such as for example, changing an alarm threshold based on personal protective equipment (e.g., PPE) that the person is wearing. In another example, the example system 2600 enables interaction with other devices to determine the situation for interaction (e.g., coupling to a smart mask to get its parameters and proper placement). The example system 2600 includes a monitoring context circuit 2602 structured to interpret a monitoring circumstance value 2604. The example system 2600 also includes a monitoring execution circuit 2606 structured to adjust a monitoring operational value 2608 in response to the monitoring circumstance value 2604. The example system 2600 includes a gas monitor 2610 having a controller 2612 that adjusts monitoring operations for a gas constituent 2614 in response to the monitoring operational value 2608.

In an embodiment, the monitoring circumstance value 2604 comprises a personal protection equipment (PPE) value 2702, and wherein the monitoring operational value 2608 comprises an alarm threshold for the gas constituent 2614. For example, the alarm level may be raised since having PPE allows for a higher compliant concentration. In other embodiments, the level could be lowered (e.g. PPE indicating a risk might be present), or the detection mix could be changed (e.g., PPE indicating additional constituents should be monitored). The PPE value 2702 includes an indicator that an operator associated with the gas monitor is wearing gas related PPE, such as a gas mask, face shield, safety glasses, flame-retardant clothing, hearing protection, work gloves, or the like. The monitoring context circuit 2602 is further structured to interpret the monitoring circumstance value 2604 in response to imaging data of an operator associated with the gas monitor. In some embodiments, the gas monitor may instruct the operator to obtain imaging data of themselves, or the gas monitor or an associated camera may obtain the imaging data. The imaging data may reveal the presence or absence of required PPE on the operator. It may also reveal improperly worn PPE, unnecessary PPE, PPE that requires additional components based on an imaged aspect of the user (e.g., operator has a beard, operator wears glasses, operator has long hair) or PPE that is out of compliance, damaged, aged, or the like.

In some embodiments, the monitoring context circuit 2602 is further structured to interpret the monitoring circumstance value in response to a communication from a smart PPE device. For example, the smart PPE device, such as a smart self-contained breathing apparatus (SCBA) device, may detect its own installation and/or proper utilization. The communication includes at least one value selected from: an indication that PPE is being worn by a user, an identifier of the PPE being worn by the user, an indication that PPE is properly secured by the user, or an indication of a compliant concentration of the gas constituent based on the PPE worn by the user. For example, the communication could indicate whether positive pressure is being applied (and how much), proper installation, operator beard, etc.

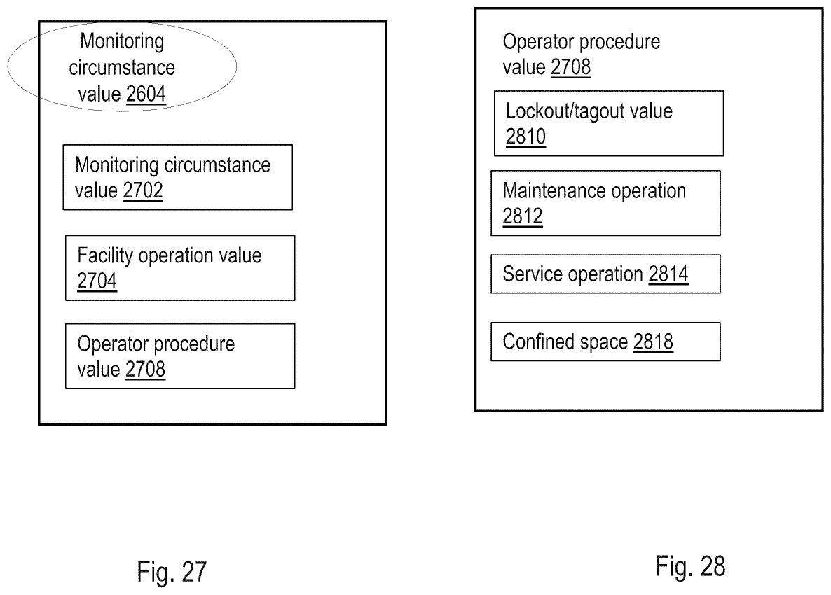

Referring to FIG. 27, the monitoring circumstance value 2604 includes a facility operation value 2704. For example, changing production operations may provide a different risk profile for gas constituents that may result in changing what is being detecting and/or the thresholds to use for alarms.

In some embodiments, the monitoring circumstance value 2604 includes an operator procedure value 2708. For example, the alarm thresholds and/or detected gases can be changed based on what the operator is doing in the moment.

Referring to FIG. 28, the operator procedure value 2708 includes at least one value selected from: a lockout/tagout (LOTO) value 2810, a maintenance operation value 2812, a service operation value 2814, or a confined space value 2818. For example, LOTO procedures, such as to prevent hazardous energy release (e.g. unexpected startup during maintenance or servicing), for some equipment may indicate the need to begin detecting certain gases and/or change the thresholds for alarm for certain gases when a LOTO value 2810 is interpreted. The LOTO value 2810 could be determined by imaging such as of an actual physical lock on a piece of equipment, of a tag on or near the equipment, of the equipment showing it to be in a particular state consistent with a LOTO operation, through digital records (e.g., records filed indicating a LOTO operation is being performed, including where in the facility and/or which personnel will be performing it), and/or text recognition from a paper form (e.g., determined from a camera or other imaging of a LOTO form being completed at the facility).

In the example system 2600, the monitoring context circuit 2602 is further structured to interpret the monitoring circumstance value 2604 in response to one of: imaging data of an operator associated with the gas monitor, a digital maintenance record, a digital service record, a digital operations record, imaging data of a facility in proximity to a user, or imaging data of a facility. For example, imaging data may detect valves near a user, and can determine what the state of equipment in the facility is (e.g., which production operations are in process) based on the imaged valves. In another example, the state of production equipment near the operator may be determined from imaging data that is not near the operator, for example, a control area may have the visual information to determine what the production state of the facility is, which may not be near the operator at all. In certain embodiments, the production operations of a facility may be determined from digital information, for example accessing a controller of the facility and/or utilizing an API to interact with facility controls to determine what operations are being performed at the facility, which can be utilized, at least in part, to determine which hazards, likely gas constituents, or the like, are present in the facility and at which locations within the facility.

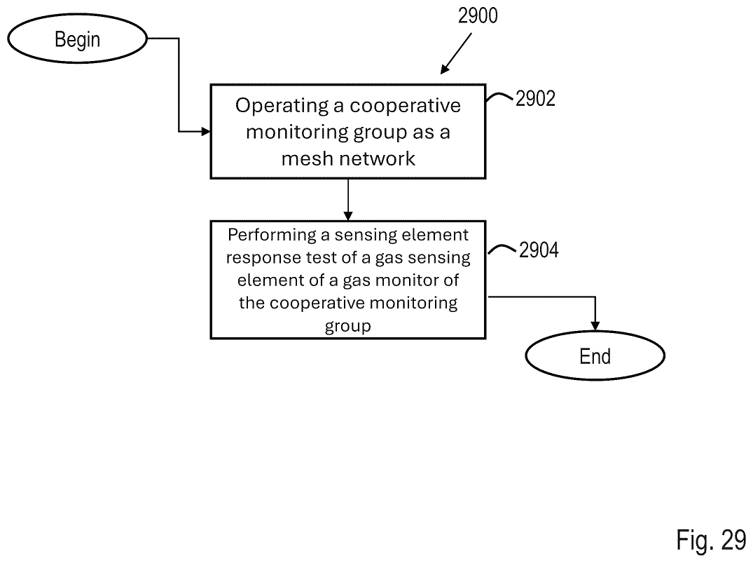

The example system 2600 is configured to perform the following operations, as depicted in the procedure 2900 shown in FIG. 29: an operation 2902 of interpreting a monitoring circumstance value, an operation 2904 of adjusting a monitoring operational value in response to the monitoring circumstance value, and an operation 2906 of adjusting monitoring operations for a gas constituent in response to the monitoring operational value. Operations also include interpreting the monitoring circumstance value in response to imaging data of an operator associated with the gas monitor or a communication from a smart PPE device. Operations also include interpreting the monitoring circumstance value in response to imaging data of an operator associated with the gas monitor. Operations also include interpreting the monitoring circumstance value in response to a digital maintenance record. Operations also include interpreting the monitoring circumstance value in response to a digital service record. Operations also include interpreting the monitoring circumstance value in response to a digital operations record. Operations also include interpreting the monitoring circumstance value in response to imaging data of a facility in proximity to a user. Operations also include interpreting the monitoring circumstance value in response to imaging data of a facility.

The methods and systems described herein may be deployed in part or in whole through a machine having a computer, computing device, processor, circuit, and/or server that executes computer readable instructions, program codes, instructions, and/or includes hardware configured to functionally execute one or more operations of the methods and systems disclosed herein. The terms computer, computing device, processor, circuit, and/or server, as utilized herein, should be understood broadly.

Any one or more of the terms computer, computing device, processor, circuit, and/or server include a computer of any type, capable to access instructions stored in communication thereto such as upon a non-transient computer readable medium, whereupon the computer performs operations of systems or methods described herein upon executing the instructions. In certain embodiments, such instructions themselves comprise a computer, computing device, processor, circuit, and/or server. Additionally or alternatively, a computer, computing device, processor, circuit, and/or server may be a separate hardware device, one or more computing resources distributed across hardware devices, and/or may include such aspects as logical circuits, embedded circuits, sensors, actuators, input and/or output devices, network and/or communication resources, memory resources of any type, processing resources of any type, and/or hardware devices configured to be responsive to determined conditions to functionally execute one or more operations of systems and methods herein.

Network and/or communication resources include, without limitation, local area network, wide area network, wireless, internet, or any other known communication resources and protocols. Example and non-limiting hardware, computers, computing devices, processors, circuits, and/or servers include, without limitation, a general purpose computer, a server, an embedded computer, a mobile device, a virtual machine, and/or an emulated version of one or more of these. Example and non-limiting hardware, computers, computing devices, processors, circuits, and/or servers may be physical, logical, or virtual. A computer, computing device, processor, circuit, and/or server may be: a distributed resource included as an aspect of several devices; and/or included as an interoperable set of resources to perform described functions of the computer, computing device, processor, circuit, and/or server, such that the distributed resources function together to perform the operations of the computer, computing device, processor, circuit, and/or server. In certain embodiments, each computer, computing device, processor, circuit, and/or server may be on separate hardware, and/or one or more hardware devices may include aspects of more than one computer, computing device, processor, circuit, and/or server, for example as separately executable instructions stored on the hardware device, and/or as logically partitioned aspects of a set of executable instructions, with some aspects of the hardware device comprising a part of a first computer, computing device, processor, circuit, and/or server, and some aspects of the hardware device comprising a part of a second computer, computing device, processor, circuit, and/or server.

A computer, computing device, processor, circuit, and/or server may be part of a server, client, network infrastructure, mobile computing platform, stationary computing platform, or other computing platform. A processor may be any kind of computational or processing device capable of executing program instructions, codes, binary instructions and the like. The processor may be or include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer readable instructions on a server, client, firewall, gateway, hub, router, or other such computer and/or networking hardware. The computer readable instructions may be associated with a server that may include a file server, print server, domain server, internet server, intranet server and other variants such as secondary server, host server, distributed server and the like. The server may include one or more of memories, processors, computer readable transitory and/or non-transitory media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, and the like. Additionally, this coupling and/or connection may facilitate remote execution of instructions across the network. The networking of some or all of these devices may facilitate parallel processing of program code, instructions, and/or programs at one or more locations without deviating from the scope of the disclosure. In addition, all the devices attached to the server through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods, program code, instructions, and/or programs may be associated with a client that may include a file client, print client, domain client, internet client, intranet client and other variants such as secondary client, host client, distributed client and the like. The client may include one or more of memories, processors, computer readable transitory and/or non-transitory media, storage media, ports (physical and virtual), communication devices, and interfaces capable of accessing other clients, servers, machines, and devices through a wired or a wireless medium, and the like. The methods, program code, instructions, and/or programs as described herein and elsewhere may be executed by the client. In addition, other devices utilized for execution of methods as described in this application may be considered as a part of the infrastructure associated with the client.

The client may provide an interface to other devices including, without limitation, servers, other clients, printers, database servers, print servers, file servers, communication servers, distributed servers, and the like. Additionally, this coupling and/or connection may facilitate remote execution of methods, program code, instructions, and/or programs across the network. The networking of some or all of these devices may facilitate parallel processing of methods, program code, instructions, and/or programs at one or more locations without deviating from the scope of the disclosure. In addition, all the devices attached to the client through an interface may include at least one storage medium capable of storing methods, program code, instructions, and/or programs. A central repository may provide program instructions to be executed on different devices. In this implementation, the remote repository may act as a storage medium for methods, program code, instructions, and/or programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules, and/or components as known in the art. The computing and/or non-computing device(s) associated with the network infrastructure may include, apart from other components, a storage medium such as flash memory, buffer, stack, RAM, ROM and the like. The methods, program code, instructions, and/or programs described herein and elsewhere may be executed by one or more of the network infrastructural elements.

The methods, program code, instructions, and/or programs described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like.

The methods, program code, instructions, and/or programs described herein and elsewhere may be implemented on or through mobile devices. The mobile devices may include navigation devices, cell phones, mobile phones, mobile personal digital assistants, laptops, palmtops, netbooks, pagers, electronic books readers, music players, and the like. These mobile devices may include, apart from other components, a storage medium such as a flash memory, buffer, RAM, ROM and one or more computing devices. The computing devices associated with mobile devices may be enabled to execute methods, program code, instructions, and/or programs stored thereon. Alternatively, the mobile devices may be configured to execute instructions in collaboration with other devices. The mobile devices may communicate with base stations interfaced with servers and configured to execute methods, program code, instructions, and/or programs. The mobile devices may communicate on a peer to peer network, mesh network, or other communications network. The methods, program code, instructions, and/or programs may be stored on the storage medium associated with the server and executed by a computing device embedded within the server. The base station may include a computing device and a storage medium. The storage device may store methods, program code, instructions, and/or programs executed by the computing devices associated with the base station.

The methods, program code, instructions, and/or programs may be stored and/or accessed on machine readable transitory and/or non-transitory media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

Certain operations described herein include interpreting, receiving, and/or determining one or more values, parameters, inputs, data, or other information. Operations including interpreting, receiving, and/or determining any value parameter, input, data, and/or other information include, without limitation: receiving data via a user input; receiving data over a network of any type; reading a data value from a memory location in communication with the receiving device; utilizing a default value as a received data value; estimating, calculating, or deriving a data value based on other information available to the receiving device; and/or updating any of these in response to a later received data value. In certain embodiments, a data value may be received by a first operation, and later updated by a second operation, as part of the receiving a data value. For example, when communications are down, intermittent, or interrupted, a first operation to interpret, receive, and/or determine a data value may be performed, and when communications are restored an updated operation to interpret, receive, and/or determine the data value may be performed.

Certain logical groupings of operations herein, for example methods or procedures of the current disclosure, are provided to illustrate aspects of the present disclosure. Operations described herein are schematically described and/or depicted, and operations may be combined, divided, re-ordered, added, or removed in a manner consistent with the disclosure herein. It is understood that the context of an operational description may require an ordering for one or more operations, and/or an order for one or more operations may be explicitly disclosed, but the order of operations should be understood broadly, where any equivalent grouping of operations to provide an equivalent outcome of operations is specifically contemplated herein. For example, if a value is used in one operational step, the determining of the value may be required before that operational step in certain contexts (e.g. where the time delay of data for an operation to achieve a certain effect is important), but may not be required before that operation step in other contexts (e.g. where usage of the value from a previous execution cycle of the operations would be sufficient for those purposes). Accordingly, in certain embodiments an order of operations and grouping of operations as described is explicitly contemplated herein, and in certain embodiments re-ordering, subdivision, and/or different grouping of operations is explicitly contemplated herein.

The methods and systems described herein may transform physical and/or or intangible items from one state to another. The methods and systems described herein may also transform data representing physical and/or intangible items from one state to another.

The elements described and depicted herein, including in flow charts, block diagrams, and/or operational descriptions, depict and/or describe specific example arrangements of elements for purposes of illustration. However, the depicted and/or described elements, the functions thereof, and/or arrangements of these, may be implemented on machines, such as through computer executable transitory and/or non-transitory media having a processor capable of executing program instructions stored thereon, and/or as logical circuits or hardware arrangements. Example arrangements of programming instructions include at least: monolithic structure of instructions; standalone modules of instructions for elements or portions thereof; and/or as modules of instructions that employ external routines, code, services, and so forth; and/or any combination of these, and all such implementations are contemplated to be within the scope of embodiments of the present disclosure Examples of such machines include, without limitation, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements described and/or depicted herein, and/or any other logical components, may be implemented on a machine capable of executing program instructions. Thus, while the foregoing flow charts, block diagrams, and/or operational descriptions set forth functional aspects of the disclosed systems, any arrangement of program instructions implementing these functional aspects are contemplated herein. Similarly, it will be appreciated that the various steps identified and described above may be varied, and that the order of steps may be adapted to particular applications of the techniques disclosed herein. Additionally, any steps or operations may be divided and/or combined in any manner providing similar functionality to the described operations. All such variations and modifications are contemplated in the present disclosure. The methods and/or processes described above, and steps thereof, may be implemented in hardware, program code, instructions, and/or programs or any combination of hardware and methods, program code, instructions, and/or programs suitable for a particular application. Example hardware includes a dedicated computing device or specific computing device, a particular aspect or component of a specific computing device, and/or an arrangement of hardware components and/or logical circuits to perform one or more of the operations of a method and/or system. The processes may be implemented in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and computer readable instructions, or any other machine capable of executing program instructions.

Thus, in one aspect, each method described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or computer-readable instructions described above. All such permutations and combinations are contemplated in embodiments of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

1.-33. (canceled)

34. A system for gas monitoring, comprising:
a cooperative monitoring group for gas monitoring, the cooperative monitoring group comprising:
a plurality of gas monitors comprising a gas sensing element and a wireless communication interface;
a gas station, comprising:
at least one gas monitor physical interface comprising a gas coupling;
a wireless communication interface; and
a test manager configured to perform a sensing element response test of the gas sensing element; and
a data manager comprising a wireless communication interface and an external communication interface; and
wherein the cooperative monitoring group is configured to operate as a mesh network, and wherein external communications of the cooperative monitoring group from the plurality of gas monitors and the gas station are routed through the data manager.

35. The system of claim 34, wherein the sensing element response test comprises a bump test.

36. The system of claim 34, wherein the gas coupling comprises a selective fluid coupling between the gas sensing element and a selected test gas.

37. The system of claim 36, wherein the test manager is further configured to provide a gas availability communication for the selected test gas to a remote device.

38. The system of claim 34, wherein the test manager is further configured to sequentially perform a plurality of sensing element response tests for a gas monitor, of the plurality of gas monitors, having a plurality of gas sensing elements.

39. The system of claim 34, wherein the sensing element response test comprises a response characteristic test.

40.-60. (canceled)
